(19) United States
(12) Patent Application Publication
(10) Pub. No.: US 2024/0226459 A9
(43) Pub. Date: Jul. 11, 2024

(54) MIXING AND INJECTION SYSTEM INCLUDING A TRACKING SENSOR

(71) Applicant: Boston Scientific Scimed Inc., Maple Grove, MN (US)

(72) Inventors: Andrew Brian Graveley, Shoreview, MN (US); Benjamin Cleveland, Bellingham, MA (US); Daniel J. Foster, Lino Lakes, MN (US); Katie Knowles, Providence, RI (US)

(73) Assignee: Boston Scientific Scimed Inc., Maple Grove, MN (US)

(21) Appl. No.: 18/491,295

(22) Filed: Oct. 20, 2023

Related U.S. Application Data
(60) Provisional application No. 63/418,152, filed on Oct. 21, 2022.

(51) Int. Cl. A61M 5/32

(52) U.S. Cl. CPC A61M 5/3294; A61B 2034/2063

(57) ABSTRACT

A medical device includes a handle having a first end and a second end, a mixer positioned within the handle and an adapter coupled to the second end of the handle. A delivery needle includes a distal end, a proximal end, and a lumen extending from the distal end to the proximal end. The proximal end of the delivery needle is coupled to the first end of the handle. A sensor cable includes a distal end and a proximal end, and the proximal end of the sensor cable is positioned within the handle, and the distal end extends distally through the lumen of the delivery needle.

### CROSS-REFERENCE TO RELATED APPLICATIONS

This application claims the benefit of U.S. Provisional Patent Application Ser. No. 63/418,152 filed Oct. 21, 2022, the disclosure of which is incorporated herein by reference.

### TECHNICAL FIELD

The present disclosure pertains to medical devices, and methods for manufacturing medical devices. More particularly, the present disclosure pertains to a medical device including a mixing and injection system having a tracking sensor integrated within and/or attached thereto.

### BACKGROUND

Needles are commonly used to deliver therapies, aspirate fluid, or acquire tissue samples, particularly in the prostate. In most cases, the needles must be guided under ultrasound, where an operator may control a two-dimensional ultrasound probe with one hand and place the needle with the other.

Placing the needle under ultrasound is difficult and requires the operator to estimate spatial distances and orientation between the ultrasound probe inside a patient and the needle outside the patient. For example, in the case of a prostate biopsy, the operator first estimates the trajectory of the needle based on the ultrasound image without any direct indication of where the needle is located relative to anatomy shown on the ultrasound image. Once the needle is inserted through a perineum, the operator aligns an ultrasound imaging plane to the needle tip in order to visualize the tip and place the needle in a desired location. However, if the needle is oblique to the ultrasound imaging plane the operator may not see the needle tip, and many operators prefer an oblique approach so they can sample all regions of the prostate through a relatively smaller area of the perineum, and thus reduce the area they need to anesthetize prior to the procedure. Once the needle is visualized and placed in the desired location, the operator then estimates the three-dimensional trajectory of the needle when the needle advances forward from the retracted position to ensure a path that the needle will travel when advanced is only through tissue that can be safely biopsied. If the needle veers towards any critical anatomy, such as the rectal wall, urethra, seminal vesicles, or blood vessels, the needle may be advanced too quickly for the operator to track or correct course, causing the critical anatomy to be pierced, leading to complications for the patient. The use of a magnetic sensor may allow the operator to track the needle inside the body and avoid such complications. However, the use of a magnetic sensor may take up valuable space within a needle and impede the injection of viscous fluids or gels, and/or limit the access of other tools. Thus, an improved medical device may be desirable.

### BRIEF SUMMARY

This disclosure provides design, material, manufacturing method, and use alternatives for medical devices. An example medical device may include a handle, the handle including a housing. The housing may include a first end and a second end, a first fluid inlet positioned near the second end of the housing, a second fluid inlet positioned near the second end of the housing, a mixing chamber in fluid communication with the first fluid inlet and the second fluid inlet, and a fluid outlet in fluid communication with the mixing chamber. The medical device may further include a delivery needle having a distal end, a proximal end, and a lumen extending from the distal end to the proximal end, wherein the proximal end of the delivery needle may be coupled to a first end of the housing and may be in fluid communication with the fluid outlet, a sensor cable having a distal end and a proximal end, wherein the proximal end of the sensor cable may be positioned within the housing of the handle, and a sensor positioned at the distal end of the sensor cable. The sensor cable may extend distally through the lumen of the delivery needle such that the sensor at the distal end of the sensor cable may be positioned near the distal end of the delivery needle.

Alternatively or additionally to any of the embodiments above, a mixer may be positioned within the mixing chamber.

Alternatively or additionally to any of the embodiments above, a first, distal valve may be positioned within the mixing chamber and a second, proximal valve may be positioned within the mixing chamber, wherein the mixer may be configured to be positioned between the first, distal valve and the second, proximal valve.

Alternatively or additionally to any of the embodiments above, a sensor channel may extend through the mixing chamber and the sensor cable may be configured to extend through the sensor channel.

Alternatively or additionally to any of the embodiments above, a plurality of rib features may be mounted on an inside wall of a distal end of the mixing chamber, the plurality of rib features extending radially inward from the inside wall and configured to hold the first, distal valve in place.

Alternatively or additionally to any of the embodiments above, an adapter may be configured to be coupled to the second end of the housing of the handle, the adapter configured to engage with the first fluid inlet and the second fluid inlet.

Alternatively or additionally to any of the embodiments above, the sensor may be a position sensor.

Alternatively or additionally to any of the embodiments above, the sensor cable may be threadably mounted within the mixing chamber.

Alternatively or additionally to any of the embodiments above, the sensor cable may be coupled to the adapter such that when the adapter is uncoupled from the second end of the housing of the handle, the sensor cable is withdrawn proximally from the delivery needle.

Alternatively or additionally to any of the embodiments above, the sensor may have an outer diameter of 0.018 inches.

Alternatively or additionally to any of the embodiments above, the delivery needle may have an outer diameter of 0.038 inches.

An example medical device may include a handle having a housing, wherein the housing may include a first end and a second end, a first fluid inlet positioned near the second end of the housing, a second fluid inlet positioned near the second end of the housing, a mixing chamber in fluid communication with the first fluid inlet and the second fluid inlet, a mixer positioned within the mixing chamber, and a fluid outlet in fluid communication with the mixing chamber. The medical device may further include a delivery needle having a distal end, a proximal end, and a lumen extending from the distal end to the proximal end, wherein the proximal end of the delivery needle may be coupled to a first end of the housing and is in fluid communication with the fluid outlet, a sensor cable having a distal end and a proximal end, wherein the proximal end of the sensor cable is positioned within the housing of the handle, and a sensor positioned at the distal end of the sensor cable. A sensor channel may extend through the mixing chamber and the sensor cable may be configured to extend through the sensor channel, and the sensor cable may extend distally through the lumen of the delivery needle such that the sensor at the distal end of the sensor cable is positioned near the distal end of the delivery needle.

Alternatively or additionally to any of the embodiments above, an adapter may be configured to be coupled to the second end of the housing of the handle, the adapter configured to engage with the first fluid inlet and the second fluid inlet.

Alternatively or additionally to any of the embodiments above, the sensor cable may be coupled to the adapter such that when the adapter is uncoupled from the second end of the housing of the handle, the sensor cable is withdrawn proximally from the delivery needle.

Alternatively or additionally to any of the embodiments above, the adapter may include a first fluid channel configured to engage with the first fluid inlet and a second fluid channel configured to engage with the second fluid inlet.

Alternatively or additionally to any of the embodiments above, a first fluid chamber may be in fluid communication with the first fluid inlet, and a second fluid chamber may be in fluid communication with the second fluid inlet.

Alternatively or additionally to any of the embodiments above, an electrical port may be positioned within the adapter and the sensor cable is operatively coupled to the electrical port.

Another example of a medical device may include a handle having a first end and a second end, a mixer positioned within the handle, an adapter coupled to the second end of the handle, a delivery needle having a distal end, a proximal end, and a lumen extending from the distal end to the proximal end, wherein the proximal end of the delivery needle is coupled to the first end of the handle, and a sensor cable having a distal end and a proximal end, wherein the proximal end of the sensor cable is positioned within the handle and the distal end extends distally through the lumen of the delivery needle.

Alternatively or additionally to any of the embodiments above, the sensor cable may be coupled to the adapter such that when the adapter is uncoupled from the second end of the housing of the handle, the sensor cable is withdrawn proximally from the delivery needle.

Alternatively or additionally to any of the embodiments above, a mixing chamber may be positioned within the handle, wherein the mixer is positioned within the mixing chamber.

The above summary of some embodiments is not intended to describe each disclosed embodiment or every implementation of the present disclosure. The Figures, and Detailed Description, which follow, more particularly exemplify these embodiments.

### BRIEF DESCRIPTION OF THE DRAWINGS

The disclosure may be more completely understood in consideration of the following detailed description in connection with the accompanying drawings, in which:

FIG. 1A illustrates an exemplary medical device including a delivery needle and an injection system;

FIG. 1B illustrates an exploded view of the exemplary medical device including the delivery needle and injection system, as in FIG. 1A;

FIG. 2A illustrates a side view of an exemplary medical device including a delivery needle, a handle, and an adapter;

FIG. 2B illustrates a bottom view of the exemplary medical device including the delivery needle, the handle, and the adapter, as in FIG. 2A;

FIG. 3A illustrates a cross-section view of the exemplary medical device including the delivery needle, the handle, and the adapter, as in FIGS. 2A and 2B, taken at line 3A-3A;

FIG. 3B illustrates an enlarged view of a distal end of the delivery needle as in FIG. 3A, taken at circle 3B;

FIG. 4 illustrates a cross-section view of the exemplary medical device including the delivery needle, the handle, and the adapter, as in FIGS. 2A and 2B, taken at line 4-4;

FIG. 5 illustrates a cross-section view of the exemplary medical device including the delivery needle, the handle, and the adapter, as in FIGS. 2A and 2B, taken at line 4-4, showing a mixer fully intact;

FIG. 6 illustrates a cross-section view of the exemplary medical device including the delivery needle, the handle, and the adapter, as in FIGS. 2A and 2B, taken at line 4-4, showing a mixer fully intact, wherein the adapter is removed from the handle;

FIG. 7A illustrates the removal of an adapter from a handle, wherein a sensor cable is removed from the handle along with the adapter;

FIG. 7B illustrates the adapter and the sensor cable, as in FIG. 7A, fully removed from the handle;

FIG. 8 illustrates an exemplary medical device including a delivery needle and an adapter;

FIG. 9 illustrates a cross-section view of the medical device including the delivery needle and the adapter, as in FIG. 8, taken at line 9-9;

FIG. 10A illustrates an exemplary medical device wherein an adapter is separate from a handle and a delivery needle; and

FIG. 10B illustrates the exemplary medical device as in FIG. 10A, wherein the adapter is connected to the handle and the delivery needle.

While the disclosure is amenable to various modifications and alternative forms, specifics thereof have been shown by way of example in the drawings and will be described in detail. It should be understood, however, that the intention is not to limit the disclosure to the particular embodiments described. On the contrary, the intention is to cover all modifications, equivalents, and alternatives falling within the spirit of the disclosure.

### DETAILED DESCRIPTION

For the following defined terms, these definitions shall be applied, unless a different definition is given in the claims or elsewhere in this specification.

All numeric values are herein assumed to be modified by the term “about,” whether or not explicitly indicated. The term “about” generally refers to a range of numbers that one of skill in the art would consider equivalent to the recited value (i.e., having the same function or result). In many instances, the term “about” may include numbers that are rounded to the nearest significant figure.

The recitation of numerical ranges by endpoints includes all numbers within that range (e.g., 1 to 5 includes, 1, 1.5, 2, 2.75, 3, 3.80, 4, and 5).

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise.

It is noted that references in this specification to “an embodiment”, “some embodiments”, “other embodiments”, etc., indicate that the embodiment described may include one or more features, structures, and/or characteristics. However, such recitations do not necessarily mean that all embodiments include the features, structures, and/or characteristics. Additionally, when features, structures, and/or characteristics are described in connection with one embodiment, such features, structures, and/or characteristics may also be used in connection with other embodiments whether or not explicitly described unless clearly stated to the contrary.

The following detailed description should be read with reference to the drawings in which similar elements in different drawings are numbered the same. The drawings, which are not necessarily to scale, depict illustrative embodiments and are not intended to limit the scope of the disclosure.

Needles are commonly used to deliver therapy, aspirate fluid, or sample tissue. In most cases, needles must be guided under ultrasound, wherein a user may control a two-dimensional ultrasound probe with one hand and place the needle with the other. Placing the needle under ultrasound may be difficult and ay require the user to estimate spatial distances and orientation between the tissue displayed in the ultrasound image and the needle as it penetrates the tissue. This coordination may be especially difficult when the ultrasound transducer is far from the access point, such as during prostate procedures.

Guiding the needle under two-dimensional ultrasound may require the user to continuously move and rotate the ultrasound probe to find the position of the needle tip and understand its trajectory. If the use fails to find and identify the needle under ultrasound, or misinterprets a partial view of the needle, the user may unintentionally pierce critical structures. Magnetic tracking offers the ability to track tool tips anywhere inside the body using a sensor. While magnetic sensors are small and may fit within the needle, the sensor may impede injection of viscous fluids or gels, or other access tools. In some cases, fluids and/or gels may need to be mixed prior to delivery into a patient. In-line static mixers may prevent the passage of a sensor into the needle. Thus, an improved medical device for delivering fluids and/or gels may be desirable.

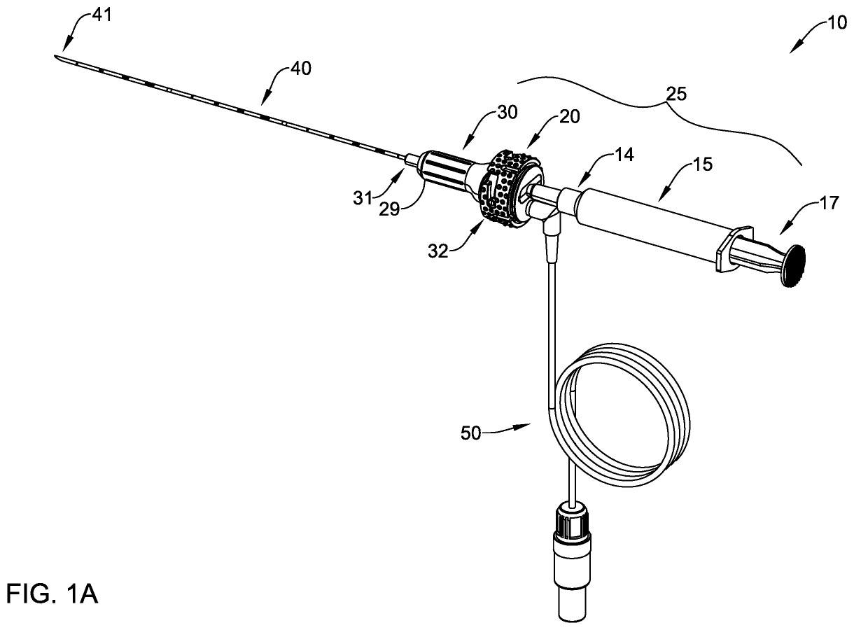

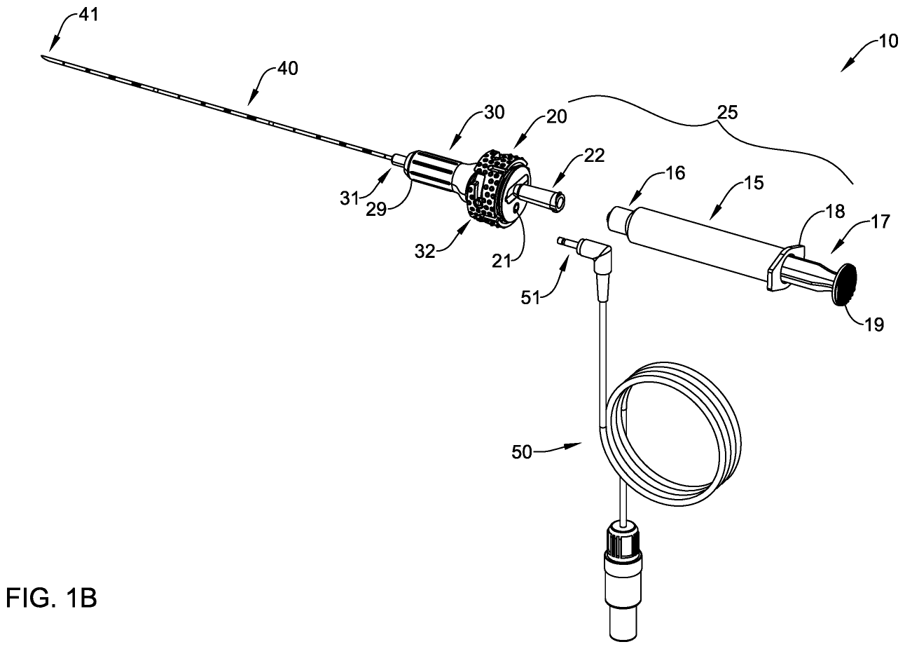

FIG. 1A illustrates an exemplary medical device 10 including a delivery needle 40 and an injection system 25. FIG. 1B illustrates an exploded view of the exemplary medical device 10 including the delivery needle 40 and the injection system 25. As shown in FIGS. 1A to 1B, the medical device 10 may be a delivery device including a handle 30, the delivery needle 40, an adapter 20, a syringe 15 and a cable 50. The handle 30 may include a housing 29 having a first end 31 and a second end 32. The delivery needle 40 may include a distal end 41 and a proximal end (not explicitly shown in FIG. 1A), and the proximal end of the delivery needle 40 may be coupled to the first end 31 of the housing 29. The delivery needle 40 may be coupled to the housing via adhesive bonding, laser welding, resistance welding, insert injection molding, or any other suitable method of attachment. In some cases, the delivery needle 40 may be beveled at the distal end 41 to enhance tissue penetration.

The adapter 20 may be removably coupled to the second end 32 of the housing 29 via a friction fit, a snap fit, or a channel lock. The syringe 15 may include a first end 16 and a second end 17. The syringe 15 may be removably coupled to the adapter 20 via a port 22. In some cases, the first end 16 of the syringe 15 may be coupled to the port 22 of the adapter 20 via a luer lock, a snap fit, an interference fit, or any other suitable method of attachment. In some cases, the syringe 15 may be configured to contain saline to, for example, flush the housing 29 and the delivery needle 40, and/or the targeted tissue, prior to delivering a therapy. In some cases, the syringe 15 may be configured to contain saline for hydro-dissection, for example. In such cases, the saline contained within the syringe 15 may be used to prime the delivery needle 40 to remove air from a lumen of the delivery needle 40 to prevent air from entering tissue and obscuring an ultrasound image. Once the delivery needle 40 is in a desired location, the saline is injected to perform hydro-dissection of the tissue. The second end 17 of the syringe 15 may include a grip 18 and a plunger 19. In use, a user may hold the grip 18 and translate the plunger 19 in a distal direction to administer a fluid (e.g., saline) contained within the syringe 15.

The cable 50 may be removably coupled to the adapter 20, via an electrical port 21. In such cases, the cable 50 may include a barrel connector 51 which may be configured to be plugged into the electrical port 21. In some cases, the cable 50 may be configured to be coupled to a controller (not shown) which may receive signals, for example, from a sensor located within the delivery needle 40, and/or transmit signals, for example, to a transmitter device.

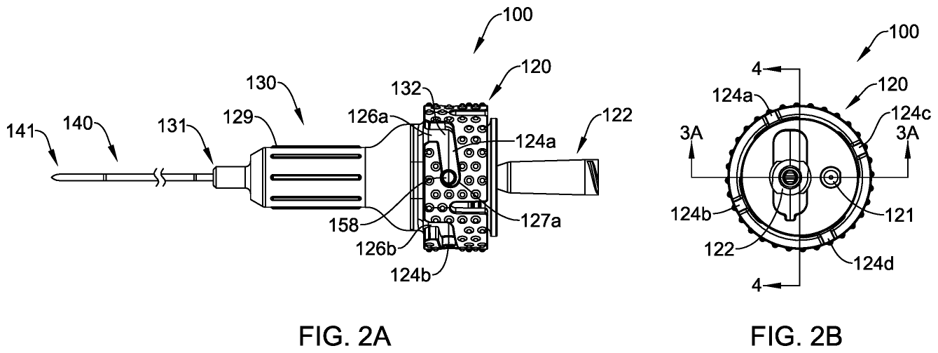

FIGS. 2A to 7B illustrate an exemplary medical device 100. FIG. 2A illustrates a side view of the exemplary medical device 100 including a delivery needle 140, a handle 130, and an adapter 120. FIG. 2B illustrates a bottom view of the exemplary medical device 100 including the delivery needle 140 (not shown in the view of FIG. 2B), the handle 130 (not shown in the view of FIG. 2B), and the adapter 120, as in FIG. 2A. The medical device 100 may be considered as an example of the medical device 10 shown in FIGS. 1A to 1B. As shown in FIGS. 2A to 2B, the medical device 100 may include the handle 130, the delivery needle 140, and the adapter 120. The handle 130 may include a housing 129 having a first end 131 and a second end 132. The delivery needle 140 may include a distal end 141 and a proximal end (not explicitly shown in FIG. 2A), and the proximal end of the delivery needle 140 may be coupled to the first end 131 of the housing 129. The delivery needle 140 may be coupled to the housing 129 via adhesive bonding, laser welding, resistance welding, insert injection molding, or any other suitable method of attachment. In some cases, the delivery needle 140 may be beveled at the distal end 141 to enhance tissue penetration.

The adapter 120 may be removably coupled to the second end 132 of the housing 129 via a channel lock. As shown in FIGS. 2A to 2B, the adapter 120 may include a plurality of channels 124a, 124b, 124c, 124c. As can be seen in FIG. 2A, the channel 124a may interact with a stop 158 on the housing 129 of the handle 130 and lock the adapter 120 onto the second end 132 of the housing 129. For example, a user may align an opening 126a of the channel 124a with the stop 158, advance the adapter 120 in a distal direction onto the second end 132 of the housing 129, and twist the adapter 120 such that the stop 158 follows the channel 124a and engages with an end 127a of the channel 124a, thereby coupling the adapter 120 to the second end 132 of the housing 129. If the user wants to uncouple the adapter 120 from the housing 129, the user may twist the adapter 120 in an opposite direction, such that the stop 158 follows the channel 124a from the end 127a of the channel 124a to the opening 126a of the channel 124a, and the user may retract the adapter 120 in a proximal direction, thereby uncoupling the adapter 120 from the housing 129. In some cases, the adapter 120 may not include the plurality of channels 124a, 124b, 124c, 124d, and may instead couple to the housing 129 via a friction fit, a snap fit, or any other suitable method of attachment.

The adapter 120 may include a port 122, which may be configured to engage with a syringe (e.g., syringe 15). The port 122 may be configured to engage with the syringe via a luer lock, an interference fit, a snap fit, or any other suitable method of engagement. The adapter may further include an electrical port 121 which may be configured to engage with a cable (e.g., cable 50). In some cases, the cable may include a barrel connector (e.g., barrel connector 51) which may be configured to be plugged into the electrical port 121.

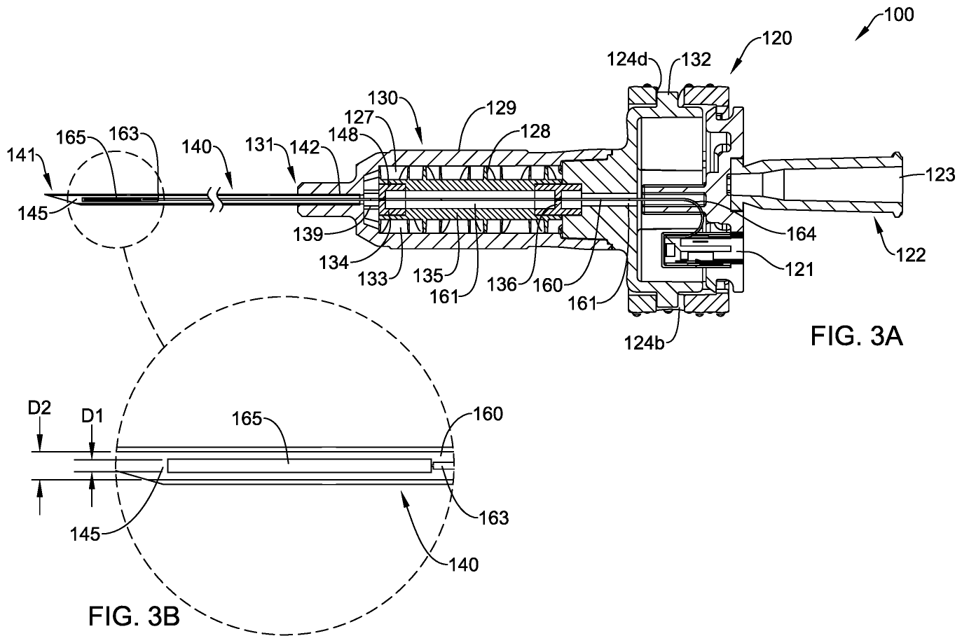

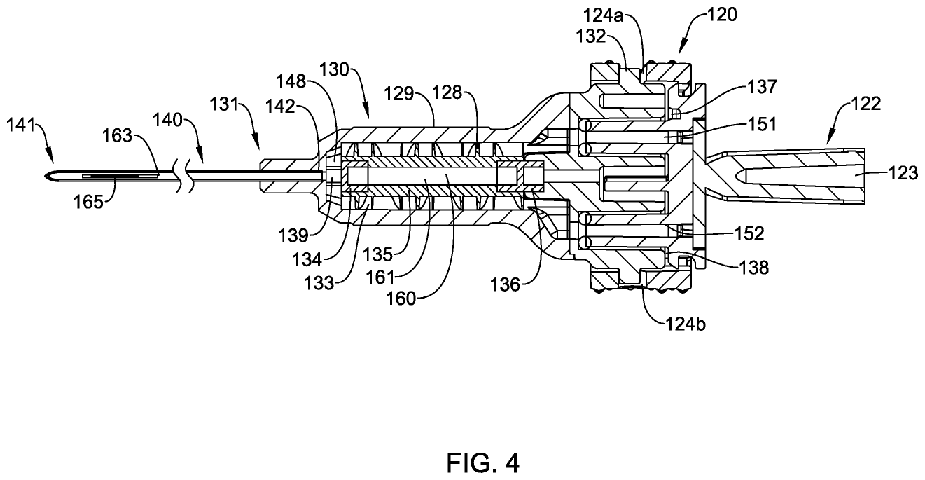

FIG. 3A illustrates a cross-section view of the exemplary medical device 100 including the delivery needle 140, the handle 130, and the adapter 120, as in FIGS. 2A and 2B, taken at line 3A-3A. As discussed with reference to FIGS. 2A and 2B, the handle 130 may include the housing 129. The housing 129 may include the first end 131 and the second end 132. The housing 129 may include a mixing chamber 133 which may be in fluid communication with a fluid outlet 139, a first fluid inlet, and a second fluid inlet. The fluid outlet 139 may be positioned near the first end 131 of the housing 129. The first fluid inlet and the second fluid inlet are shown in FIG. 4. A mixer 135 may be positioned within the mixing chamber 133 between the fluid outlet 139 and the first and second fluid inlets. The mixer 135 may be a static mixer, such as, for example, a ribbon mixer, an in-line mixer, and or any other suitable static mixer. In some cases, the mixer 135 may be a non-static mixer that includes moving parts, however this is not shown. The mixer 135 may include a plurality of blades 128 that extend into the mixing chamber 133. The plurality of blades 128 are stationary, providing a stop for a fluid injected into the mixing chamber 133, thereby causing the injected fluids to mix together within the mixing chamber 133. In some cases, there may be one type of fluid injected into the mixing chamber 133, such as saline, for example. In some cases, there may be more than one type of fluid injected into the mixing chamber 133, such as water and polyethylene glycol (PEG), for example.

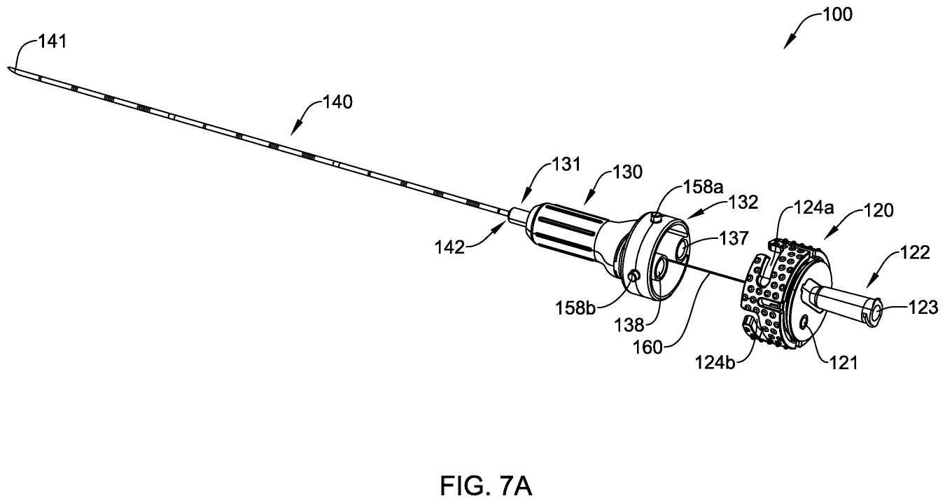

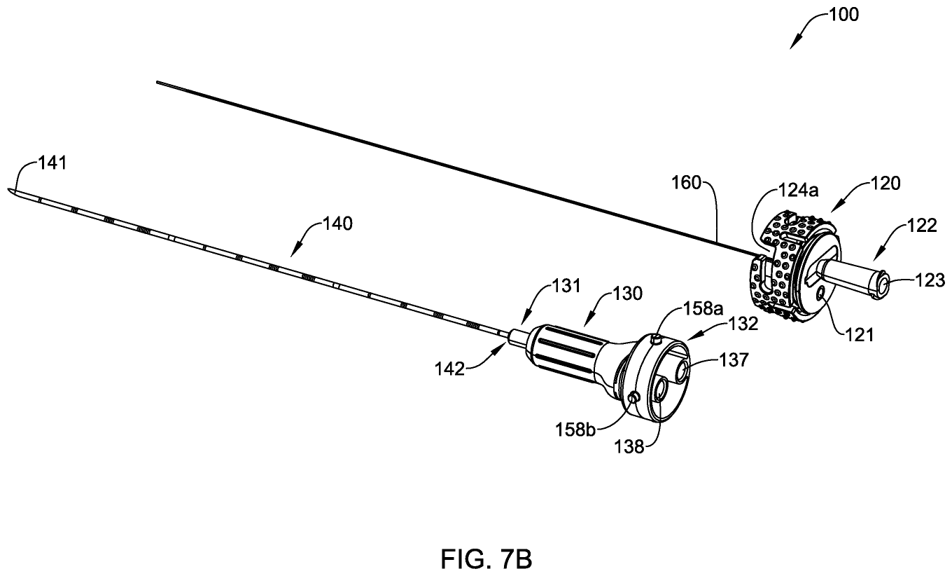

A sensor cable 160 may include a distal end 163 and a proximal end 164. The proximal end 164 of the sensor cable 160 may be positioned within the handle 130 and the distal end 163 may extend distally through a lumen 145 of the delivery needle 140. The proximal end 164 of the sensor cable 160 may be operatively connected via a thin, flex circuit, to the electrical port 121 in the adapter 120. A sensor channel 161 may extend from the adapter 120 through the housing 129 and the mixer 135 within the mixing chamber 133. The sensor cable 160 may be configured to extend from the electrical port 121 in the adapter 120, through the sensor channel 161, and distally through the lumen 145 of the delivery needle 140. In some cases, the sensor cable 160 may be threadably mounted within the sensor channel 161. In some cases, the sensor cable 160 may be coupled to the adapter 120 such that when the adapter 120 is uncoupled from the second end 132 of the housing 129, the sensor cable 160 is withdrawn proximally from the lumen 145 of the delivery needle 140 and the sensor channel 161 within the mixer 135, as illustrated in FIGS. 7A to 7B.

A sensor 165 may be positioned at the distal end 163 of the sensor cable 160. In some cases, the sensor 165 may be a position sensor, such as an electromagnetic sensor or an optical sensor. The sensor 165 may enable a position and/or an orientation of a distal end 141 of the delivery needle 140 to be tracked. In some cases, the sensor 165 may facilitate tracking of a position and/or an orientation of the distal end 141 of the delivery needle 140 relative to an ultrasound imaging plane such that the position and/or the orientation of the delivery needle 140 may be displayed in an imaging plane, although this is not shown. The distal end 141 of the delivery needle 140 including the sensor 165 will be described in further detail with reference to FIG. 3B.

A first, distal valve 134 and a second, proximal valve 136 may be positioned within the mixing chamber 133, and the mixer 135 may be positioned between the first, distal valve 134 and the second, proximal valve 136. The first, distal valve 134 and the second, proximal valve 136 may be configured to provide a seal around the mixer 135, thereby preventing a fluid and/or air from entering or exiting the mixer 135, respectively. Providing the sensor channel 161 within the mixer 135 allows the sensor cable 160 and the sensor 165 to pass through the mixer 135 within the mixing chamber 133 and into the lumen 145 of the delivery needle 140 without obstructing any fluids and/or air being delivered to a patient via the mixing chamber 133. The mixing chamber 133 may include a plurality of rib features 148 mounted on an inside wall 127 of the mixing chamber 133. The plurality of rib features 148 may be positioned near the first end 131 of the housing 129. The plurality of rib features 148 may be configured to extend radially inward from the inside wall 127, and may be configured to hold the first, distal valve 134 in place. By holding the first, distal valve 134 in place, fluid is allowed to flow through the mixing chamber 133, around the mixer 135 and into the lumen 145 of the delivery needle 140.

As previously stated, the delivery needle 140 includes the distal end 141 and the proximal end 142, and the lumen 145 extending from the distal end 141 to the proximal end 142. The proximal end 142 may be coupled to the first end 131 of the housing 129. The proximal end 142 of the delivery needle 140 may be configured to fit within the first end 131 of the housing 129, and may be secured to the housing 129 via adhesive bonding, laser welding, resistance welding, insert injection molding, or any, other suitable method of attachment. The lumen 145 of the delivery needle 140 may be in fluid communication with the fluid outlet 139 of the housing 129.

As described above with reference to FIGS. 2A and 2B, the adapter 120 may be removably coupled to the second end 132 of the housing 129. The adapter 120 may include the port 122, which may be configured to engage with a syringe (e.g., syringe 15). The port 122 may be configured to engage with the syringe via a luer lock, an interference fit, a snap fit, or any other suitable method of engagement. The port 122 may include a port lumen 123. The port lumen 123 may be configured to engage with a first fluid inlet and a second fluid inlet, as will be further described with reference to FIG. 4. The adapter may further include the electrical port 121 which may be configured to engage with a cable (e.g., cable 50). In some cases, the cable may include a barrel connector (e.g., barrel connector 51) which may be configured to be plugged into the electrical port 121.

FIG. 3B illustrates an enlarged view of the distal end 141 of the delivery needle 140 as in FIG. 3A, taken at circle 3B. As shown in FIG. 3B, the distal end 141 of the delivery needle 140 may be beveled to enhance tissue penetration. The delivery needle 140 may include the lumen 145, and the sensor cable 160 may extend distally through the lumen 145 of the delivery needle 140 such that the sensor 165 may be positioned near the distal end 141 of the delivery needle 140. In some cases, the delivery needle 140 may include an outer diameter in a range of about 0.012 inches to 0.180 inches. In some cases, the delivery needle 140 may include an outer diameter in a range of about 0.030 inches to 0.075 inches. In some cases, the delivery needle 140 may include an outer diameter D₁ of 0.038 inches. In some cases, the sensor 165 may include an outer diameter in a range of about 0.005 inches to 0.149 inches. In some cases, the sensor 165 may include a diameter in a range of about 0.015 inches to 0.071 inches. In some cases, the sensor 165 may include an outer diameter D2 of 0.018 inches.

FIG. 4 illustrates a cross-section view of the exemplary medical device including the delivery needle 140, the handle 130, and the adapter 120, as in FIGS. 2A and 2B, taken at line 4-4. The cross-section view in FIG. 4 shows a first fluid inlet 137 positioned near the second end 132 of the housing 129, and a second fluid inlet 138 positioned near the second end 132 of the housing 129. The adapter 120 may be configured to engage with the first fluid inlet 137 and the second fluid inlet 138. For example, the adapter 120 includes a first fluid chamber 151 in fluid communication with the first fluid inlet 137, and a second fluid chamber 152 in fluid communication with the second fluid inlet 138. In some cases, the first fluid chamber 151 may extend distally within the first fluid inlet 137 and the second fluid chamber 152 may extend distally within the second fluid inlet 138. In use, a user may inject a fluid through the port lumen 123 of the port 122. The fluid may then flow through the first fluid chamber 151 and the second fluid chamber 152 into the first fluid inlet 137 and the second fluid inlet 138, respectively, and subsequently into the mixing chamber 133. The fluid then flows from the mixing chamber 133 into the lumen 145 of the delivery needle 140 and into the tissue of a patient.

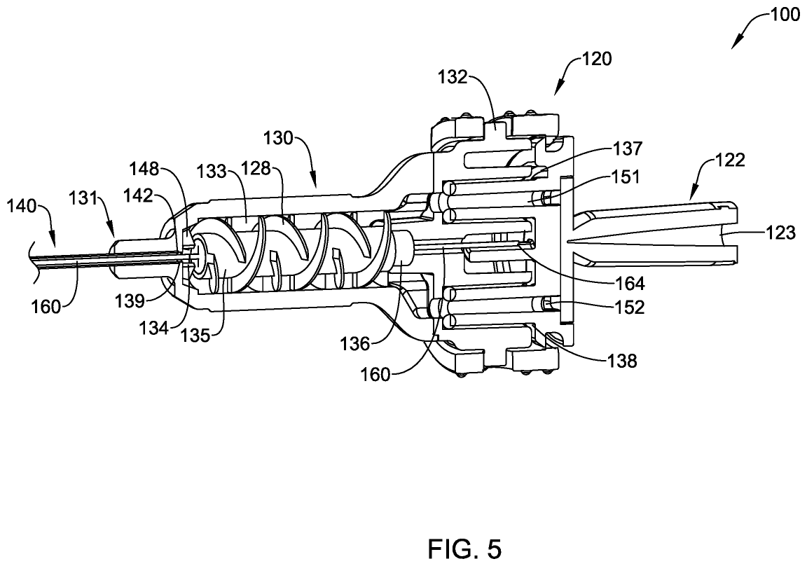

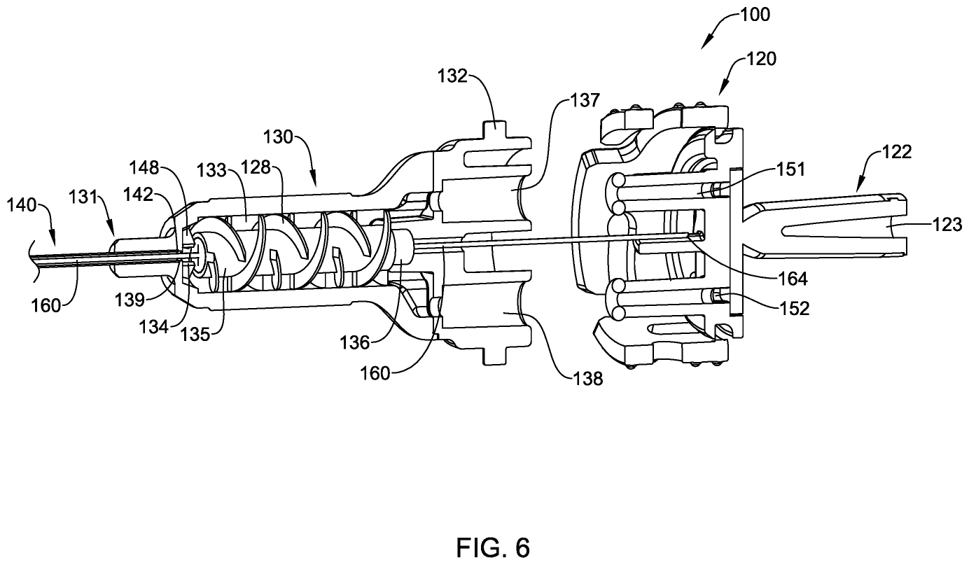

FIG. 5 illustrates a cross-section view of the exemplary medical device 100 including the delivery needle 140, the handle 130, and the adapter 120, as in FIGS. 2A and 2B, taken at line 4-4, showing the mixer 135 fully intact. FIG. 6 illustrates a cross-section view of the exemplary medical device including the delivery needle 140, the handle 130, and the adapter 120, as in FIGS. 2A and 2B, taken at line 4-4, showing a mixer fully intact, wherein the adapter 120 is removed from the handle 130. As shown in FIGS. 5 to 6, the mixer 135 may be positioned within the mixing chamber 133 between the fluid outlet 139 and the first fluid inlet 137 and the second fluid inlet 138. The mixer 135 may be a static mixer, such as, for example, a ribbon mixer, an in-line mixer, and or any other suitable static mixer. In some cases, the mixer 135 may be a non-static mixer that includes moving parts, however this is not shown. The mixer 135 may include the plurality of blades 128 that extend into the mixing chamber 133. The plurality of blades 128 are stationary, providing a stop for a fluid injected into the mixing chamber 133, thereby causing the injected fluids to mix together within the mixing chamber 133. In some cases, there may be one type of fluid injected into the mixing chamber 133, such as saline, for example. In some cases, there may be more than one type of fluid injected into the mixing chamber 133, such as water and polyethylene glycol (PEG), for example.

Upon removal of the adapter 120 from the handle 130, the adapter 120 is retracted proximally from the handle 130. When the adapter 120 is retracted proximally, the first fluid chamber 151 is disengaged from the first fluid inlet 137, and a second fluid chamber 152 is disengaged from the second fluid inlet 138. As shown in FIG. 6, the sensor cable 160 is coupled to the adapter 120, thus, the sensor cable 160 is retracted proximally from the delivery needle 140 and the handle 130 when the adapter 120 is disengaged from the handle 130.

FIG. 7A illustrates the removal of the adapter 120 from the handle 130, wherein the sensor cable 160 is retracted proximally from the delivery needle 140 and the handle 130, via removal of the adapter 120. FIG. 7B illustrates the adapter 120 and the sensor cable 160, as in FIG. 7A, fully removed from the delivery needle 140 and the handle 130. As previously discussed, with reference to FIGS. 2A to 2B, the adapter 120 may be removably coupled to the second end 132 of the housing 129 via a channel lock. The adapter 120 may include the plurality of channels 124a, 124b, 124c, 124c, although only channels 124a, 124b can be seen in FIGS. 7A to 7B. When the user uncouples the adapter 120 from the housing 129, the user may twist the adapter 120 in direction such that a stop 158a follows the channel 124a from the end (e.g., end 127a) of the channel 124a to the opening (e.g., opening 126a) of the channel 124, and the stop 158b follows the channel 124b from a first end of the channel 124b to an opening of the channel 124b, and the user may retract the adapter 120 in a proximal direction, thereby uncoupling the adapter 120 from the housing 129. In some cases, the adapter 120 may not include the plurality of channels 124a, 124b, 124c, 124d, and may instead couple to the housing 129 via a friction fit, a snap fit, or any other suitable method of attachment.

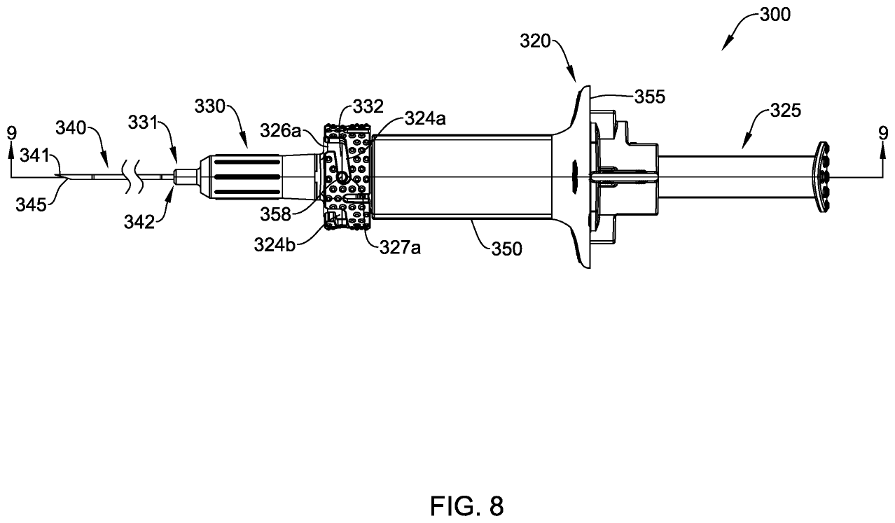

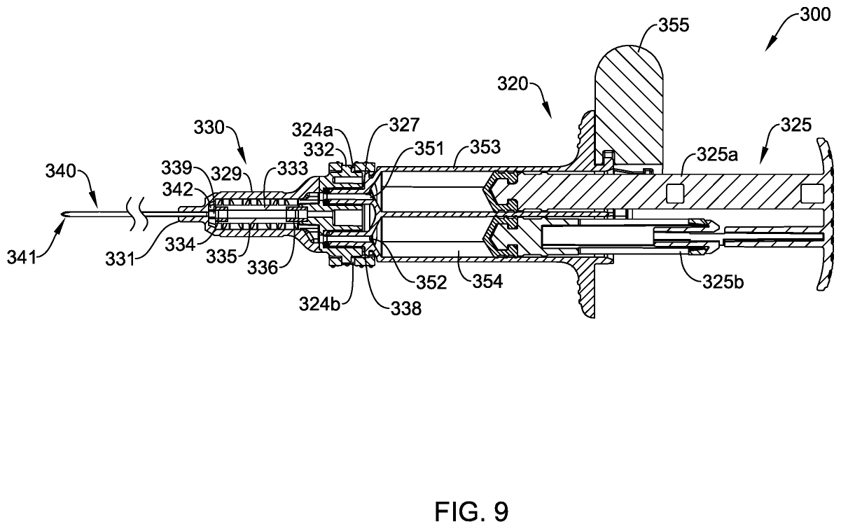

FIG. 8 illustrates an exemplary medical device 300 including a delivery needle 340 and an adapter 320. FIG. 9 illustrates a cross-section view of the medical device 300 including the delivery needle 340 and the adapter 320, as in FIG. 8, taken at line 9-9. The medical device 300 may include a handle 330, the delivery needle 340, and the adapter 320. The handle 330 may include a housing 329 having a first end 331 and a second end 332. The delivery needle 340 may include a distal end 341 and a proximal end 342, and a lumen 345 extending from the distal end 341 to the proximal end 342. The proximal end 342 of the delivery needle 340 may be coupled to the first end 331 of the housing 329. The delivery needle 340 may be coupled to the housing 329 via adhesive bonding, laser welding, resistance welding, insert injection molding, or any other suitable method of attachment. In some cases, the delivery needle 340 may be beveled at the distal end 341 to enhance tissue penetration.

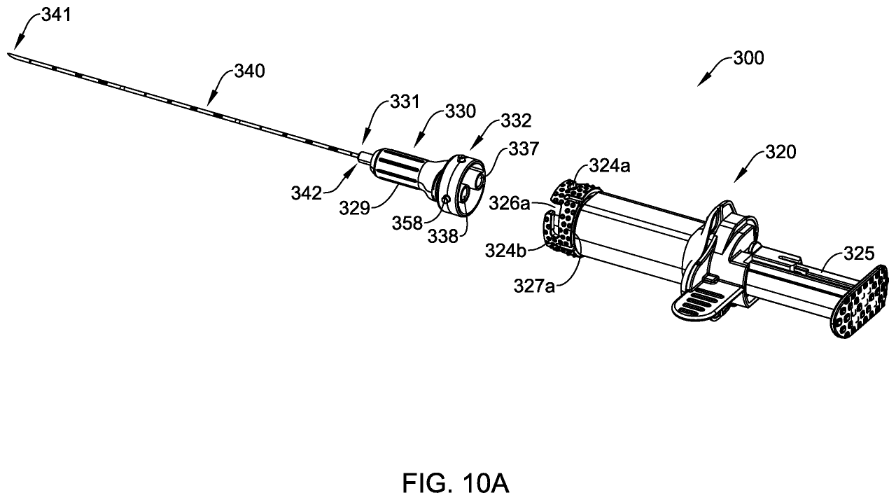

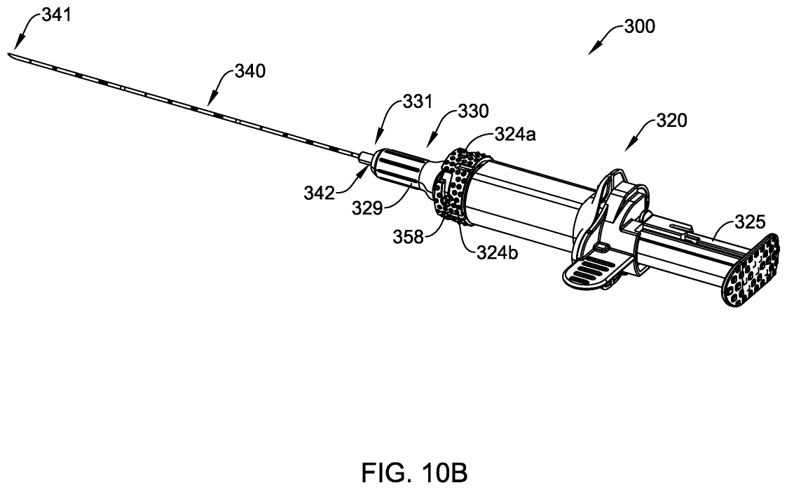

The adapter 320 may be removably coupled to the second end 332 of the housing 329 via a channel lock, as will be further shown with reference to FIG. 10A. As shown in FIGS. 8 to 9, the adapter 320 may include a plurality of channels 324a, 324b. As can be seen in FIGS. 8 to 9, the channel 324a may interact with a stop 358 on the housing 329 of the handle 330 and lock the adapter 320 onto the second end 332 of the housing 329. For example, a user may align an opening 326a of the channel 324a with the stop 358, advance the adapter 320 in a distal direction onto the second end 332 of the housing 329, and twist the adapter 320 such that the stop 358 follows the channel 324a and engages with an end 327a of the channel 324a, thereby coupling the adapter 320 to the second end 332 of the housing 329. If the user wants to uncouple the adapter 320 from the housing 329, the user may twist the adapter 320 in an opposite direction, such that the stop 358 follows the channel 324a from the end 327a of the channel 324a to the opening 326a of the channel 324, and the user may retract the adapter 320 in a proximal direction, thereby uncoupling the adapter 320 from the housing 329. In some cases, the adapter 320 may not include the plurality of channels 324a, 324b and may instead couple to the housing 329 via a friction fit, a snap fit, or any other suitable method of attachment.

The adapter 320 may include a barrel 350, a grip 355, and a plunger 325. As shown in FIG. 9, in some cases, the adapter 320 may be a double barrel syringe, and the barrel 350 may include a first inner barrel 353 and a second inner barrel 354. In some cases, the adapter 320 may be a single barrel syringe. The plunger 325 may include a first plunger 325a configured to engage with the first inner barrel 353 and a second plunger 325b configured to engage with the second inner barrel 354. The first inner barrel 353 may be configured to engage with a first fluid chamber 351 within the adapter 320. The second inner barrel 354 may be configured to engage with the second fluid chamber 352.

The housing 329 may include a first fluid inlet 337 positioned near the second end 332 of the housing 329, and a second fluid inlet 338 positioned near the second end 332 of the housing 329. The adapter 320 may be configured to engage with the first fluid inlet 337 and the second fluid inlet 338. For example, the first fluid chamber 351 may be in fluid communication with the first fluid inlet 337, and the second fluid chamber 352 may be in fluid communication with the second fluid inlet 338. In some cases, the first fluid chamber 351 may extend distally within the first fluid inlet 337 and the second fluid chamber 352 may extend distally within the second fluid inlet 338. In use, the first inner barrel 353 may include a first fluid (e.g., water), and the second inner barrel 354 may include a second fluid (e.g., PEG). A user may deploy the plunger 325 in a distal direction which may inject the first fluid into the first fluid chamber 351 and through the first fluid inlet 337, and the second fluid into the second fluid chamber 352 and through the second fluid inlet 338. The fluid may then flow through the first fluid chamber 351 and the second fluid chamber 352 into the first fluid inlet 337 and the second fluid inlet 338, respectively, and subsequently into a mixing chamber 333. The fluid then flows from the mixing chamber 333 into the lumen 345 of the delivery needle 340 and into the tissue of a patient.

As shown in FIGS. 8 and 9, the handle 330 may include the housing 329. The housing 329 may include the first end 331 and the second end 332. The housing 329 may include the mixing chamber 333 which may be in fluid communication with a fluid outlet 339, the first fluid inlet 337, and the second fluid inlet 338. The fluid outlet 339 may be positioned near the first end 331 of the housing 329. A mixer 335 may be positioned within the mixing chamber 333 between the fluid outlet 339 and the first fluid inlet 337 and second fluid inlet 338. The mixer 335 may be a static mixer, such as, for example, a ribbon mixer, an in-line mixer, and or any other suitable static mixer. In some cases, the mixer 335 may be a non-static mixer that includes moving parts, however this is not shown. The mixer 335 may include a plurality of blades 328 that extend into the mixing chamber 333. The plurality of blades 328 are stationary, providing a stop for a fluid injected into the mixing chamber 333, thereby causing the injected fluids to mix together within the mixing chamber 333. In some cases, there may be one type of fluid injected into the mixing chamber 333, such as saline, for example. In some cases, there may be more than one type of fluid injected into the mixing chamber 333, such as water and polyethylene glycol (PEG), for example.

A first, distal valve 334 and a second, proximal valve 336 may be positioned within the mixing chamber 333, and the mixer 335 may be positioned between the first, distal valve 334 and the second, proximal valve 336. The first, distal valve 334 and the second, proximal valve 336 may be configured to provide a seal around the mixer 335, thereby preventing a fluid and/or air from entering or exiting the mixer 335, respectively.

FIG. 10A illustrates the exemplary medical device 300 wherein the adapter 320 is separate from the handle 330 and the delivery needle 340, and FIG. 10B illustrates the exemplary medical device 300 as in FIG. 10A, wherein the adapter 320 is connected to the handle 330 and the delivery needle 340. The medical device 300 may be a delivery device including the handle 330, the delivery needle 340, and the adapter 320. As previously discussed, the adapter 320 may be a double barrel syringe. The handle 330 may include the housing 329 having the first end 331 and the second end 332. The delivery needle 340 may include the distal end 341 and a proximal end (not explicitly shown in FIGS. 10A and 10B), and the proximal end of the delivery needle 340 may be coupled to the first end 331 of the housing 329.

The adapter 320 may be removably coupled to the second end 332 of the housing 329 via a friction fit, a snap fit, or a channel lock. As shown in FIGS. 10A to 10B, the adapter 320 may include the plurality of channels 324a, 324b. As can be seen in FIG. 10B, the channel 324a may interact with the stop 358 on the housing 329 of the handle 330 and lock the adapter 320 onto the second end 332 of the housing 329. For example, a user may align the opening 326a of the channel 324a with the stop 358, advance the adapter 320 in a distal direction onto the second end 332 of the housing 329, and twist the adapter 320 such that the stop 358 follows the channel 324a and engages with the end 327a of the channel 324a, thereby coupling the adapter 320 to the second end 332 of the housing 329. If the user wants to uncouple the adapter 320 from the housing 329, the user may twist the adapter 320 in an opposite direction, such that the stop 358 follows the channel 324a from the end 327a of the channel 324a to the opening 326a of the channel 324, and the user may retract the adapter 320 in a proximal direction, thereby uncoupling the adapter 320 from the housing 329. In some cases, the adapter 320 may not include the plurality of channels 324a, 324b and may instead couple to the housing 329 via a friction fit, a snap fit, or any other suitable method of attachment.

In some cases, the adapter 320 may be configured to contain saline to, for example, flush the housing 329 and the delivery needle 340, and/or the targeted tissue, prior to delivering a therapy. In some cases, the adapter 320 may be configured to contain saline for hydro-dissection, for example. In some cases, the adapter 320 may be configured to contain multiple fluids, such as water and PEG. In such cases, when a user translates the plunger 325 in a distal direction, the water, and the PEG flow into the mixing chamber 333 within the housing 329 where the two fluids mix prior to being injected into a patient via the delivery needle 340.

The medical device 10, 100, 300, and/or various parts thereof, may be made from a metal, metal alloy, polymer (some examples of which are disclosed below), a metal-polymer composite, ceramics, combinations thereof, and the like, or other suitable material. Some examples of suitable polymers may include polytetrafluoroethylene (PTFE), ethylene tetrafluoroethylene (ETFE), fluorinated ethylene propylene (FEP), polyoxymethylene (POM, for example, DELRIN® available from DuPont), polyether block ester, polyurethane (for example, Polyurethane 85A), polypropylene (PP), polyvinylchloride (PVC), polyether-ester (for example, ARNITEL® available from DSM Engineering Plastics), ether or ester based copolymers (for example, butylene/poly(alkylene ether) phthalate and/or other polyester elastomers such as HYTREL® available from DuPont), polyamide (for example, DURETHAN® available from Bayer or CRISTAMID® available from Elf Atochem), elastomeric polyamides, block polyamide/ethers, polyether block amide (PEBA, for example available under the trade name PEBAX®), ethylene vinyl acetate copolymers (EVA), silicones, polyethylene (PE), MARLEX® high-density polyethylene, MARLEX® low-density polyethylene, linear low density polyethylene (for example REXELL®), polyester, polybutylene terephthalate (PBT), polyethylene terephthalate (PET), polytrimethylene terephthalate, polyethylene naphthalate (PEN), polyetheretherketone (PEEK), polyimide (PI), polyetherimide (PEI), polyphenylene sulfide (PPS), polyphenylene oxide (PPO), poly paraphenylene terephthalamide (for example, KEVLAR®), polysulfone, nylon, nylon-12 (such as GRILAMID® available from EMS American Grilon), perfluoro(propyl vinyl ether) (PFA), ethylene vinyl alcohol, polyolefin, polystyrene, epoxy, polyvinylidene chloride (PVdC), poly(styrene-b-isobutylene-b-styrene) (for example, SIBS and/or SIBS 50A), polycarbonates, ionomers, biocompatible polymers, other suitable materials, or mixtures, combinations, copolymers thereof, polymer/metal composites, and the like. In some embodiments the sheath can be blended with a liquid crystal polymer (LCP). For example, the mixture can contain up to about 6 percent LCP.

In at least some embodiments, portions, or all of the medical device 10, 100, 300 may also be doped with, made of, or otherwise include a radiopaque material. Radiopaque materials are understood to be materials capable of producing a relatively bright image on a fluoroscopy screen or another imaging technique during a medical procedure. This relatively bright image aids the user of medical device 10, 100, 300 in determining its location. Some examples of radiopaque materials can include, but are not limited to, gold, platinum, palladium, tantalum, tungsten alloy, polymer material loaded with a radiopaque filler, and the like. Additionally, other radiopaque marker bands and/or coils may also be incorporated into the design of medical device 10, 100, 300 to achieve the same result.

This disclosure is, in many respects, only illustrative. Changes may be made in details, particularly in matters of shape, size, and arrangement of steps without exceeding the scope of the disclosure. This may include, to the extent that it is appropriate, the use of any of the features of one example embodiment being used in other embodiments. The disclosure's scope is, of course, defined in the language in which the appended claims are expressed.

What is claimed is:

1. A medical device comprising:
a body, comprising:
a housing, the housing having a first end and a second end;
at least one fluid inlet positioned near the second end of the housing; and
a chamber in fluid communication with the at least one fluid inlet; and
a fluid outlet in fluid communication with the chamber;
a delivery needle having a distal end, a proximal end, and a lumen extending from the distal end to the proximal end, wherein the proximal end of the delivery needle is coupled to a first end of the housing and is in fluid communication with the fluid outlet;
a sensor cable having a distal end and a proximal end, wherein the proximal end of the sensor cable is positioned within the housing; and
a sensor positioned at the distal end of the sensor cable;
wherein the sensor cable extends distally through the lumen of the delivery needle such that the sensor at the distal end of the sensor cable is positioned near the distal end of the delivery needle.

2. The medical device of claim 1, wherein the chamber is a mixing chamber, the device further comprising a mixer is positioned within the mixing chamber.

3. The medical device of claim 2, wherein a first, distal valve is positioned within the mixing chamber and a second, proximal valve is positioned within the mixing chamber, wherein the mixer is configured to be positioned between the first, distal valve and the second, proximal valve.

4. The medical device of claim 3, wherein a sensor channel extends through the mixer and the sensor cable is configured to extend through the sensor channel.

5. The medical device of claim 3, wherein a plurality of rib features are mounted on an inside wall of the mixing chamber, the plurality of rib features extending radially inward from the inside wall and configured to hold the first, distal valve in place.

6. The medical device of claim 1, wherein the at least one fluid inlet comprises a first fluid inlet and a second fluid inlet, the medical device further comprising: an adapter configured to be coupled to the second end of the housing, the adapter configured to engage with the first fluid inlet and the second fluid inlet.

7. The medical device of claim 1, wherein the sensor is a position sensor.

8. The medical device of claim 4, wherein the sensor cable is threadably mounted within the sensor channel.

9. The medical device of claim 6, wherein the sensor cable is coupled to the adapter such that when the adapter is uncoupled from the second end of the housing, the sensor cable is withdrawn proximally from the delivery needle.

10. The medical device of claim 1, wherein the sensor has an outer diameter in a range of 0.005 inches to 0.149 inches.

11. The medical device of claim 1, wherein the delivery needle has an outer diameter in a range of 0.012 inches to 0.180 inches.

12. A medical device comprising:
a handle, the handle comprising:
a housing, the housing having a first end and a second end;
a first fluid inlet positioned near the second end of the housing;
a second fluid inlet positioned near the second end of the housing;
a mixing chamber in fluid communication with the first fluid inlet and the second fluid inlet;
a mixer positioned within the mixing chamber; and
a fluid outlet in fluid communication with the mixing chamber;
a delivery needle having a distal end, a proximal end, and a lumen extending from the distal end to the proximal end, wherein the proximal end of the delivery needle is coupled to a first end of the housing and is in fluid communication with the fluid outlet;
a sensor cable having a distal end and a proximal end, wherein the proximal end of the sensor cable is positioned within the housing of the handle; and
a sensor positioned at the distal end of the sensor cable;
wherein a sensor channel extends through the mixer and the sensor cable is configured to extend through the sensor channel; and
wherein the sensor cable extends distally through the lumen of the delivery needle such that the sensor at the distal end of the sensor cable is positioned near the distal end of the delivery needle.

13. The medical device of claim 12 further comprising: an adapter configured to be coupled to the second end of the housing of the handle, the adapter configured to engage with the first fluid inlet and the second fluid inlet.

14. The medical device of claim 13, wherein the sensor cable is coupled to the adapter such that when the adapter is uncoupled from the second end of the housing of the handle, the sensor cable is withdrawn proximally from the delivery needle.

15. The medical device of claim 13, wherein the adapter includes a first fluid channel configured to engage with the first fluid inlet and a second fluid channel configured to engage with the second fluid inlet.

16. The medical device of claim 13, wherein an electrical port is positioned within the adapter and the sensor cable is operatively coupled to the electrical port.

17. The medical device of claim 12, wherein a first fluid chamber in in fluid communication with the first fluid inlet, and a second fluid chamber is in fluid communication with the second fluid inlet.

18. A medical device comprising:
a handle having a first end and a second end;
a mixer positioned within the handle;
an adapter coupled to the second end of the handle;
a delivery needle having a distal end, a proximal end, and a lumen extending from the distal end to the proximal end, wherein the proximal end of the delivery needle is coupled to the first end of the handle; and
a sensor cable having a distal end and a proximal end, wherein the proximal end of the sensor cable is positioned within the handle and the distal end extends distally through the lumen of the delivery needle.

19. The medical device of claim 18, wherein the sensor cable is coupled to the adapter such that when the adapter is uncoupled from the second end of the housing, the sensor cable is withdrawn proximally from the delivery needle.

20. The medical device of claim 18 further comprising: a mixing chamber positioned within the handle, wherein the mixer is positioned within the mixing chamber.
